(19) United States
(12) Patent Application Publication
(10) Pub. No.: US 2026/0355177 A1
(43) Pub. Date: Nov. 12, 2026

(54) TREATMENT FOR APLASTIC ANEMIA (AA)

(71) Applicant: ZYDUS LIFESCIENCES LIMITED, Ahmedabad, Gujarat (IN)

(72) Inventors: Mukul Jain, Ahmedabad (IN); Amit Joharapurkar, Ahmedabad (IN); Hardikkumar Savsani, Ahmedabad (IN)

(21) Appl. No.: 19/099,976

(22) PCT Filed: Jul. 31, 2023
(86) PCT No.: PCT/IB2023/057752

§ 371 Date: Jan. 30, 2025

(30) Foreign Application Priority Data
Aug. 1, 2022 (IN) 202221043866

(51) Int. Cl. A61K 31/4704; A61K 31/4152; A61K 31/426; A61K 31/496; A61K 38/19; A61P 7/06

(52) U.S. Cl. CPC A61K 31/4704; A61K 31/4152; A61K 31/426; A61K 31/496; A61K 38/196; A61P 7/06

(57) ABSTRACT

The present invention describes a combination of a suitable prolyl hydroxylase inhibitors with a suitable thrombopoietin (TPO) receptor agonist for the treatment of Aplastic anemia (AA). The invention also describes the preparation of such combination and its compositions.

### FIELD OF THE INVENTION

The present invention describes a combination of a suitable prolyl hydroxylase inhibitors with a suitable thrombopoietin (TPO) receptor agonist for the treatment of Aplastic anemia (AA). The invention also describes the preparation of such combination and its compositions.

### BACKGROUND OF THE INVENTION

Aplastic anemia (AA) is a rare disorder characterized by suppression of bone marrow function resulting in progressive pancytopenia.¹ The most common cause of AA is immune system attacking the stem cells in bone marrow. Radiation and chemotherapy treatments are the other factors that can injure bone marrow and affect blood cell production.² It is diagnosed with values have to lower than 10 g/dl for hemoglobin, 1.5×10⁹/L for neutrophils and 50×10⁹/L for platelets.³ Megakaryocytes pattern may be useful in distinguishing myelodysplasia (MDS) from AA since they are often reduced/absent in AA whereas small or aberrant megakaryocytes are more typical of MDS.⁴ The clinical consequences are anemia, usually with a requirement for frequent red blood cell transfusions, life-threatening bleeding from thrombocytopenia, and infection as a result of neutropenia. Bacterial and fungal infections are significant cause of morbidity and mortality.⁵

The standard definitive therapy for treating aplastic anemia is hematopoietic stem cell transplantation for young and medically fit patients with suitable donors and immunosuppressive therapy (IST) with antithymocyte globulin (ATG) and cyclosporine. Thrombopoietin mimetic (TPO agonist), has recently emerged as an encouraging and promising agents for patients with AA. This class has been demonstrated to be efficacious in restoring trilineage hematopoiesis. thrombopoietin (TPO) receptor agonist is the main regulator for platelet production and its receptor (c-Mpl) is present on megakaryocytes and hematopoietic stem cells.⁶ thrombopoietin (TPO) receptor agonist are intravenous peptibody Romiplostim, oral small molecules Eltrombopag, Avatrombopag and lusutrombopag.⁷

Eltrombopag bisethanolamine, chemical name 3′-(2-(1-(3,4-dimethylphenyl)-3-methyl-5-oxo-1,5-dihydro-4H-pyrazol-4-ylidene)hydrazineyl)-2′-hydroxy-[1,1′-biphenyl]-3-carboxylic acid 2-hydroxy-N,N,N-trimethylethan-1-aminium (1:1).

Eltrombopag is a small-molecule, non-peptide thrombopoietin (TPO) receptor agonist that has been shown to stimulate the proliferation and differentiation of megakaryocytes, the bone marrow cells that give rise to blood platelets in pre-clinical research and clinical trials.

Eltrombopag bisethanolamine is the active ingredient of the marketed drug Promacta® (US) or Revolade® (EU) indicated in the treatment of chronic immune thrombocytopenia (CIT) in patients who have had an insufficient response to corticosteroids, immunoglobulins, or splenectomy. It is also indicated for thrombocytopenia in patients with chronic hepatitis C and in severe aplastic anaemia. Eltrombopag is disclosed in U.S. Pat. No. 7,160,870. Eltrombopag bisethanolamine salt is disclosed in U.S. Pat. No. 7,547,719. Eltrombopag tromethamine salt is disclosed in International Patent Publication No. WO20170042839. Crystalline forms of Eltrombopag choline are disclosed in International Patent Publication No. WO20190071111 and U.S. Pat. No. 11,072,586.

Romiplostim is an active ingredient of the marketed drug Nplate. It is a thrombopoietin receptor agonist indicated for the treatment of thrombocytopenia in patients with chronic immune thrombocytopenia (ITP) who have had an insufficient response to corticosteroids, immunoglobulins, or splenectomy. Romiplostim disclosed in U.S. Pat. No. 9,145,450 and WO2000/24770.

The small molecule hypoxia-inducible factor stabilizer compound Desidustat (compound of formula (Ia)), inhibits the prolyl hydroxylase and has demonstrated hematinic potential by combined effects on endogenous erythropoietin release and efficient iron utilization. It also enhances erythroid maturation by suppressing hepcidin-ferroportin axis.⁸

Several other prolyl hydroxylase inhibitors have been disclosed in EP661269, WO2007070359, WO2008076425, WO2011007856, WO2012106472, and WO2013043621. Specifically WO2004108681 and WO2008002576 covers the prolyl hydroxylase inhibitors named Roxadustat and Vadadustat respectively.

WO2014102818 discloses compounds of the following general formula

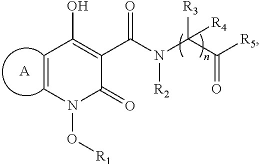

These compounds are reported to be useful for the treatment of anemia. It has surprisingly now been found that compound of formula (Ia) as given below:

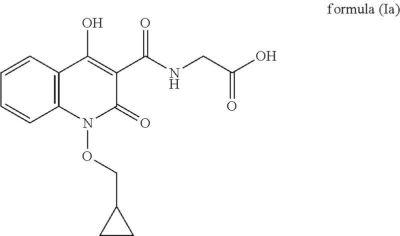 [SMILES: O=C(O)CNC(=O)c1c(O)c2ccccc2n(OCC2CC2)c1=O]

and its pharmaceutically acceptable salts are effective in the treatment of aplastic anemia when combine with a suitable TPO receptor agonist improve two lineage of hematopoietic precursor cells for the benefit of patients. Also, such combination with cyclosporine will be useful.

### Embodiments of the Invention

The present invention describes a combination of a suitable prolyl hydroxylase inhibitor or its pharmaceutically acceptable salts and a suitable thrombopoietin (TPO) receptor agonist.

The present invention describes a combination of the compound of formula (Ia) or its pharmaceutically acceptable salts and a suitable thrombopoietin (TPO) receptor agonist.

In an embodiment, the present invention provides a combination of the compound of formula (Ia) or its pharmaceutically acceptable salts and a suitable thrombopoietin (TPO) receptor agonist for the treatment of Aplastic anemia (AA).

In a further embodiment is provided a pharmaceutical composition comprising, the compound of formula (Ia) or its pharmaceutically acceptable salts and a suitable thrombopoietin (TPO) receptor agonist along with at least one a suitable pharmaceutically acceptable carrier, diluents, vehicle or excipient for the treatment of Aplastic anemia (AA).

In another embodiment, present invention provides use of combination of the compound of formula (Ia) or its pharmaceutically acceptable salts and a suitable thrombopoietin (TPO) receptor agonist for the treatment of Aplastic anemia (AA).

In an embodiment, the present invention provides a method of treating Aplastic anemia (AA) using the compound of formula (Ia) or its pharmaceutically acceptable salts.

In an embodiment the present invention provides a combination of the compound of formula (Ia) or its pharmaceutically acceptable salts and a suitable thrombopoietin (TPO) receptor agonist optionally with cyclosporine.

### BRIEF DESCRIPTION OF DRAWINGS

FIG. 1. The effect of formula (Ia), Romiplostim and their combination on white blood cells (A) and Platelets (B) in busulphan-induced aplastic anemia in Balb/c mice.

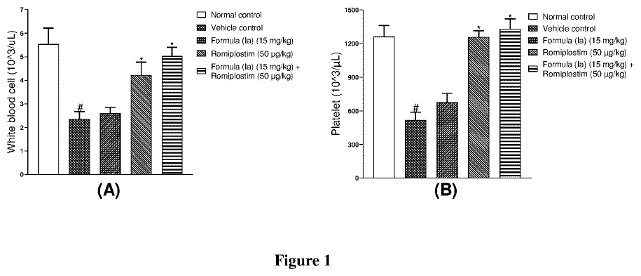

FIG. 2. The effect of formula (Ia) and their combination with Romiplostim on white blood cells (A) and Platelets (B) in cyclophosphamide-induced aplastic anemia in C57 mice.

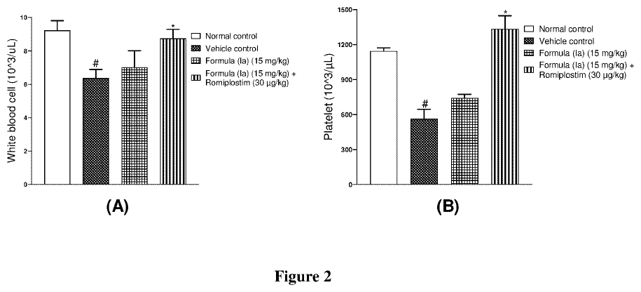

### SUMMARY OF THE INVENTION

The present invention describes a combination of a suitable prolyl hydroxylase inhibitors with a suitable thrombopoietin (TPO) receptor agonist for the treatment of Aplastic anemia (AA). The invention also describes the preparation of such combination and its compositions.

### DESCRIPTION OF THE INVENTION

### Definition

The terms ‘treatment’ or ‘treat’ refer to slowing, stopping, or delaying the progression of the disease or clinical symptoms in a patient, as evidenced by a decrease or elimination of a clinical or diagnostic symptom of the disease, disorder or condition. The term ‘subject’ refer to a human and animal. The term ‘effective amount’ in the context of the administration of the amount of the drug substance sufficient to have the desired effect. The term ‘pharmaceutically acceptable’ use embraces both human and veterinary use. The compound of formula (Ta) is Desidustat.

Combination of a Suitable Prolyl Hydroxylase Inhibitors with a Suitable Thrombopoietin (TPO) Receptor Agonist

In an embodiment, the present invention describes a combination of a suitable prolyl hydroxylase inhibitors and suitable thrombopoietin (TPO) receptor agonist.

A suitable prolyl hydroxylase inhibitor is selected from Roxadustat, Vadadustat, Daprodustat, Molidustat, Enardustat and the compound of formula (Ia) or its pharmaceutically acceptable salts.

A suitable thrombopoietin (TPO) receptor agonist is Romiplostim or its pharmaceutically acceptable salts, Eltrombopag or its pharmaceutically acceptable salts, Avatrombopag or its pharmaceutically acceptable salts and Lusutrombopag or its pharmaceutically acceptable salts.

Combination of Compound of Formula (Ia) or its Pharmaceutically Acceptable Salts with a Suitable Thrombopoietin (TPO) Receptor Agonist

In an embodiment, present invention provides a combination of compound of formula (Ia) or its pharmaceutically acceptable salts with a suitable thrombopoietin (TPO) receptor agonist, wherein formula (Ia) is represented by:

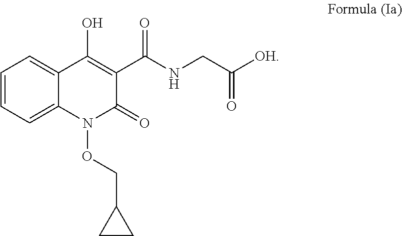 [SMILES: O=C(O)CNC(=O)c1c(O)c2ccccc2n(OCC2CC2)c1=O]

The pharmaceutical acceptable salts of the compound of formula (Ia) may be selected from suitable inorganic metal salts or organic amines salts.

The inorganic metal salt may be selected from calcium, sodium, potassium, lithium, barium, strontium, magnesium, cesium, copper, cobalt, iron, manganese, lead, aluminum, cadmium, silver, zinc, ammonium and the like.

Organic amines salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, alanine, lysine, arginine, histidine, threonine, proline, glutamine, glycine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N—N-diethylethanolamine, deanol, hydrabamine, betaine, adamantanamine, L-adamantanmethylamine, tritylamine, glucamine, N-methyl pyrrolidine, urea, procaine, metformin, hexane-1-6-diamine, 2-(2-aminoethoxy)ethanamine, N-methylmorpholine, and N-ethylmorpholine.

A suitable thrombopoietin (TPO) receptor agonist is Romiplostim or its pharmaceutically acceptable salts, Eltrombopag or its pharmaceutically acceptable salts, Avatrombopag or its pharmaceutically acceptable salts and Lusutrombopag or its pharmaceutically acceptable salts.

### Modes of Administration

Combination as described above can be administered as oral, rectal, topical, nasal, pulmonary, ocular, intestinals or parenteral.

An effective amount, e.g., dose, of combination compound or drug can readily be determined by routine experimentation, as can an effective and convenient route of administration and an appropriate formulation. Various formulations and drug delivery systems are available in the art. (See, e.g., Gennaro, ed. (2000) Remington's Pharmaceutical Sciences; and Hardman, Limbird, and Gilman, eds. (2001) The Pharmacological Basis of Therapeutics, supra.)

A suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

In certain embodiments, the compound of formula (Ia) or its pharmaceutically acceptable salts, for oral administration to a subject at a dose in the range of 1 mg to 500 mg or in the range of 50 mg to 450 mg or in the range of 100 mg to 400 mg or in the range of 150 mg to 350 mg or in the range of 200 mg to 300 mg or in the range of 1 mg to 50 mg or in the range of 1 mg to 25 mg to the subject. In certain embodiments, the compound of formula (Ia) or a pharmaceutically acceptable salts for oral administration to a subject to provide the compound of formula (Ta) or a pharmaceutically acceptable salts at a dose of 1 mg to 150 mg. In further embodiments, the compound of formula (Ia) or its pharmaceutically acceptable salts, for oral administration to a subject at a dose in the range of 1 mg to 2.5 mg or 2.5 mg to 5 mg or 5 mg to 7.5 mg or 7.5 mg to 10 mg or 10 mg to 12.5 mg or 12.5 mg to 15 mg or 15 mg to 17.5 mg or 17.5 mg to 20 mg or 20 mg to 22.5 mg or 22.5 mg to 25 mg or 25 mg to 30 mg or 30 mg to 35 mg or 35 mg to 40 mg or 40 mg to 45 mg or 45 mg to 50 mg or 50 mg to 55 mg or 55 mg to 60 mg or 60 mg to 65 mg or 65 mg to 70 mg or 70 mg to 75 mg or 75 mg to 80 mg or 80 mg to 85 mg or 85 mg to 90 mg or 90 mg to 95 mg or 95 mg to 100 mg.

In a further embodiment, Eltrombopag is administered orally to a subject in an amount of about 1 mg to about 500 mg. In a further embodiment, Avatrombopag is administered orally to a subject in an amount of about 1 mg to 100 mg. In a further embodiment, Lusutrombopag is administered orally to a subject in an amount of about 1 mg to 100 mg.

In a further embodiment, Eltrombopag is administered orally to a subject in an amount of about 12.5 mg, 25 mg, 50 mg, 75 mg and 100 mg. In a further embodiment, Avatrombopag is administered orally to a subject in an amount of about 20 mg. In a further embodiment, Lusutrombopag is administered orally to a subject in an amount of about 3 mg.

In certain embodiments, the compound of formula (Ia) or its pharmaceutically acceptable salts, for parenteral administration to a subject at a dose in the range of 1 mg to 50 mg. In a further embodiments, the compound of formula (Ia) or its pharmaceutically acceptable salts, for parenteral administration to a subject at a dose in the range of 1 mg to 2.5 mg or 2.5 mg to 5 mg or 5 mg to 7.5 mg or 7.5 mg to 10 mg or 10 mg to 12.5 mg or 12.5 mg to 15 mg or 15 mg to 17.5 mg or 17.5 mg to 20 mg or 20 mg to 22.5 mg or 22.5 mg to 25 mg or 27.5 mg to 30 mg or 30 mg to 32.5 mg or 32.5 mg to 35 mg or 35 mg to 37.5 mg or 37.5 mg to 40 mg or 40 mg to 42.5 mg or 42.5 mg to 45 mg or 45 mg to 47.5 mg or 47.5 mg to 50 mg. In certain embodiments, the compound of formula (Ia) or a pharmaceutically acceptable salts for parenteral administration to a subject to provide the compound of formula (Ia) or a pharmaceutically acceptable salts at a dose of 0.5 mg, 1 mg, 1.5 mg, 2 mg, 2.5 mg, 3 mg, 2.5 mg, 4 mg, 4.5 mg, 5 mg, 5.5 mg, 6 mg, 6.5 mg, 7 mg, 7.5 mg, 8 mg, 8.5 mg, 9 mg, 9.5 mg and 10 mg to the subject.

In a further embodiment, Romiplostim is administered parenterally to a subject in an amount of about 1 μg to about 500 μg, about 50 μg to about 450 μg, about 100 μg to about 400 μg, about 150 μg to about 350 μg, about 200 μg to about 300 μg.

In a certain embodiment, Romiplostim is administered parenterally to a subject in an amount of about 125 μg, 250 μg and 500 μg. In a further embodiment.

In certain embodiments, combination of compounds of formula (Ia) or its pharmaceutically acceptable salts and a suitable thrombopoietin (TPO) receptor agonist can be further characterized according to the dose of compound administered to a subject, where subject is animal or human.

In some embodiments, compounds of formula (Ia) or its pharmaceutically acceptable salts and the a suitable thrombopoietin (TPO) receptor agonist are administered as one composition. In some embodiments, compounds of formula (Ia) or its pharmaceutically acceptable salts and a suitable thrombopoietin (TPO) receptor agonist are administered separately. In some embodiments, compounds of formula (Ia) or its pharmaceutically acceptable salts and a suitable thrombopoietin (TPO) receptor agonist are administered consecutively. In some embodiments, compounds of formula (Ia) or its pharmaceutically acceptable salts and the a suitable thrombopoietin (TPO) receptor agonist are administered simultaneously.

Method of Treating Aplastic Anemia (AA) in Combination of Compound of Formula (Ia) or its Pharmaceutically Acceptable Salts with a Suitable Thrombopoietin (TPO) Receptor Agonist

In an embodiment, the present invention provides a method of treating Aplastic anemia (AA) in a subject, comprising administering a combination of compound of formula (Ia) or its pharmaceutically acceptable salts with a suitable thrombopoietin (TPO) receptor agonist.

The pharmaceutical acceptable salts of the compound of formula (Ia) may be selected from a suitable inorganic metal salts or organic amines salts.

The inorganic metal salt may be selected from calcium, sodium, potassium, lithium, barium, strontium, magnesium, cesium, copper, cobalt, iron, manganese, lead, aluminum, cadmium, silver, zinc, ammonium and the like.

Organic amines salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, alanine, lysine, arginine, histidine, threonine, proline, glutamine, glycine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N—N-diethylethanolamine, deanol, hydrabamine, betaine, adamantanamine, L-adamantanmethylamine, tritylamine, glucamine, N-methyl pyrrolidine, urea, procaine, metformin, hexane-1-6-diamine, 2-(2-aminoethoxy)ethanamine, N-methylmorpholine, and N-ethylmorpholine.

A suitable thrombopoietin (TPO) receptor agonist is Romiplostim or its pharmaceutically acceptable salts, Eltrombopag or its pharmaceutically acceptable salts, Avatrombopag or its pharmaceutically acceptable salts and Lusutrombopag or its pharmaceutically acceptable salts.

Combination as described above can be administered as oral, rectal, topical, nasal, pulmonary, ocular, intestinal or parenteral.

In certain embodiments, the compound of formula (Ia) or its pharmaceutically acceptable salts, for oral administration to a subject at a dose in the range of 1 mg to 500 mg or in the range of 50 mg to 450 mg or in the range of 100 mg to 400 mg or in the range of 150 mg to 350 mg or in the range of 200 mg to 300 mg or in the range of 1 mg to 50 mg or in the range of 1 mg to 25 mg to the subject. In certain embodiments, the compound of formula (Ia) or a pharmaceutically acceptable salts for oral administration to a subject to provide the compound of formula (Ta) or a pharmaceutically acceptable salts at a dose of 1 mg to 150 mg. In further embodiments, the compound of formula (Ia) or its pharmaceutically acceptable salts, for oral administration to a subject at a dose in the range of 1 mg to 2.5 mg or 2.5 mg to 5 mg or 5 mg to 7.5 mg or 7.5 mg to 10 mg or 10 mg to 12.5 mg or 12.5 mg to 15 mg or 15 mg to 17.5 mg or 17.5 mg to 20 mg or 20 mg to 22.5 mg or 22.5 mg to 25 mg or 25 mg to 30 mg or 30 mg to 35 mg or 35 mg to 40 mg or 40 mg to 45 mg or 45 mg to 50 mg or 50 mg to 55 mg or 55 mg to 60 mg or 60 mg to 65 mg or 65 mg to 70 mg or 70 mg to 75 mg or 75 mg to 80 mg or 80 mg to 85 mg or 85 mg to 90 mg or 90 mg to 95 mg or 95 mg to 100 mg.

In a further embodiment, Eltrombopag is administered orally to a subject in an amount of about 1 mg to about 500 mg. In a further embodiment, Avatrombopag is administered orally to a subject in an amount of about 1 mg to 100 mg. In a further embodiment, Lusutrombopag is administered orally to a subject in an amount of about 1 mg to 100 mg.

In a further embodiment, Eltrombopag is administered orally to a subject in an amount of about 12.5 mg, 25 mg, 50 mg, 75 mg and 100 mg. In a further embodiment, Avatrombopag is administered orally to a subject in an amount of about 20 mg. In a further embodiment, Lusutrombopag is administered orally to a subject in an amount of about 3 mg.

In certain embodiments, the compound of formula (Ia) or its pharmaceutically acceptable salts, for parenteral administration to a subject at a dose in the range of 1 mg to 50 mg. In a further embodiments, the compound of formula (Ia) or its pharmaceutically acceptable salts, for parenteral administration to a subject at a dose in the range of 1 mg to 2.5 mg or 2.5 mg to 5 mg or 5 mg to 7.5 mg or 7.5 mg to 10 mg or 10 mg to 12.5 mg or 12.5 mg to 15 mg or 15 mg to 17.5 mg or 17.5 mg to 20 mg or 20 mg to 22.5 mg or 22.5 mg to 25 mg or 27.5 mg to 30 mg or 30 mg to 32.5 mg or 32.5 mg to 35 mg or 35 mg to 37.5 mg or 37.5 mg to 40 mg or 40 mg to 42.5 mg or 42.5 mg to 45 mg or 45 mg to 47.5 mg or 47.5 mg to 50 mg. In certain embodiments, the compound of formula (Ia) or a pharmaceutically acceptable salts for parenteral administration to a subject to provide the compound of formula (Ia) or a pharmaceutically acceptable salts at a dose of 0.5 mg, 1 mg, 1.5 mg, 2 mg, 2.5 mg, 3 mg, 2.5 mg, 4 mg, 4.5 mg, 5 mg, 5.5 mg, 6 mg, 6.5 mg, 7 mg, 7.5 mg, 8 mg, 8.5 mg, 9 mg, 9.5 mg and 10 mg to the subject.

In a further embodiment, Romiplostim is administered parenterally to a subject in an amount of about 1 μg to about 500 μg, about 50 μg to about 450 μg, about 100 μg to about 400 μg, about 150 μg to about 350 μg, about 200 μg to about 300 μg.

In a certain embodiment, Romiplostim is administered parenterally to a subject in an amount of about 125 μg, 250 μg and 500 μg. In a further embodiment.

In certain embodiments, combination of compounds of formula (Ia) or its pharmaceutically acceptable salts and a suitable thrombopoietin (TPO) receptor agonist can be further characterized according to the dose of compound administered to a subject, where subject is animal or human.

In some embodiments, compounds of formula (Ia) or its pharmaceutically acceptable salts and the a suitable thrombopoietin (TPO) receptor agonist are administered as one composition. In some embodiments, compounds of formula (Ia) or its pharmaceutically acceptable salts and a suitable thrombopoietin (TPO) receptor agonist are administered separately. In some embodiments, compounds of formula (Ia) or its pharmaceutically acceptable salts and a suitable thrombopoietin (TPO) receptor agonist are administered consecutively. In some embodiments, compounds of formula (Ia) or its pharmaceutically acceptable salts and the a suitable thrombopoietin (TPO) receptor agonist are administered simultaneously.

### Pharmaceutical Composition of Compound of Formula (Ia) or its Pharmaceutically Acceptable Salts and a Suitable Thrombopoietin (TPO) Receptor Agonist for Use in Treating Aplastic Anemia (AA)

In an embodiment, present invention provides a pharmaceutical composition comprising compound of formula (Ia) or its pharmaceutically acceptable salts and a suitable thrombopoietin (TPO) receptor agonist optionally with one or more pharmaceutically acceptable excipients for use in treating Aplastic anemia (AA).

The pharmaceutical acceptable salts of the compound of formula (Ia) may be selected from suitable inorganic metal salts or organic amines salts.

The inorganic metal salt may be selected from calcium, sodium, potassium, lithium, barium, strontium, magnesium, cesium, copper, cobalt, iron, manganese, lead, aluminum, cadmium, silver, zinc, ammonium and the like.

Organic amines salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, alanine, lysine, arginine, histidine, threonine, proline, glutamine, glycine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N—N-diethylethanolamine, deanol, hydrabamine, betaine, adamantanamine, L-adamantanmethylamine, tritylamine, glucamine, N-methyl pyrrolidine, urea, procaine, metformin, hexane-1-6-diamine, 2-(2-aminoethoxy)ethanamine, N-methylmorpholine, and N-ethylmorpholine.

A suitable thrombopoietin (TPO) receptor agonist is Romiplostim or its pharmaceutically acceptable salts, Eltrombopag or its pharmaceutically acceptable salts, Avatrombopag or its pharmaceutically acceptable salts and Lusutrombopag or its pharmaceutically acceptable salts.

The pharmaceutical composition as mentioned in above embodiment may be in form of oral, rectal, topical, nasal, pulmonary, ocular, intestinal, or parenteral.

### Oral Formulation:

The pharmaceutical composition is in the oral form.

The pharmaceutically oral composition as described above or anywhere in the specification may be in the form of tablet, capsule and oral liquids.

The pharmaceutically acceptable excipients are selected at least one from diluent, binders, disintegrating agents, lubricating agents, glidant agent, sweetener, pH modifier, suspending agent or viscosity modifying agent, flavouring agent and optionally coating redimix.

Diluents include, but are not limited to lactose monohydrate, lactose, microcrystalline cellulose, polymethacrylates selected from Eudragit, potassium chloride, sulfobutylether b-cyclodextrin, sodium chloride, spray dried lactose, and preferably sulfobutyl ether b-cyclodextrin combinations thereof and other such materials known to those of ordinary skill in the art.

Binders include, but are not limited to hypromellose 3 Cps, carbomers selected from carbopol, gellan, gum Arabic, hydrogenated vegetable oil, polymethacrylates selected from Eudragit, xanthan, lactose and Zein combinations thereof and other such materials known to those of ordinary skill in the art.

Disintegrating agents include, but are not limited to, croscarmellose Sodium, bicarbonate salt, chitin, gellan gum, polacrillin potassium and docusate Sodium combinations thereof and other such materials known to those of ordinary skill in the art.

Glidant agents include, but are not limited to, colloidal silica, calcium silicate, magnesium silicate, silicon hydrogel, cornstarch, talc, combinations thereof and other such materials known to those of ordinary skill in the art.

Lubricant agents include, but are not limited to, calcium stearate, magnesium stearate, mineral oil, stearic acid, zinc stearate, glycerin behenate, hydrogenated vegetable oil, sodium stearyl fumarate and myristic Acid suitable combinations thereof and other such materials known to those of ordinary skill in the art.

A suitable pH modifying agents which maintain the pH of the formulation according to the present invention include, but are not limited to Citric acid and other similar excipients and their suitable combinations and other materials known to those of ordinary skill in the art.

Suspending agents or viscosity agent according to the present invention include, but are not limited to microcrystalline cellulose and carboxymethylcellulose sodium (Avicel CL 611) and other similar excipients and their suitable combinations and other materials known to those of ordinary skill in the art.

Sweetener is selected from Sucrose and all such materials known to those of ordinary skill in the art.

Flavouring agent is selected from cherry flavour, orange flavour, mango flavour and all such fruit flavour known to those of ordinary skill in the art.

Coating redimix is selected from Opadry Pink all such materials known to those of ordinary skill in the art.

In certain embodiments, the compound of formula (Ia) or its pharmaceutically acceptable salts, for oral administration to a subject at a dose in the range of 1 mg to 500 mg or in the range of 50 mg to 450 mg or in the range of 100 mg to 400 mg or in the range of 150 mg to 350 mg or in the range of 200 mg to 300 mg or in the range of 1 mg to 50 mg or in the range of 1 mg to 25 mg to the subject. In certain embodiments, the compound of formula (Ia) or a pharmaceutically acceptable salts for oral administration to a subject to provide the compound of formula (Ia) or a pharmaceutically acceptable salts at a dose of 1 mg to 150 mg. In further embodiments, the compound of formula (Ia) or its pharmaceutically acceptable salts, for oral administration to a subject at a dose in the range of 1 mg to 2.5 mg or 2.5 mg to 5 mg or 5 mg to 7.5 mg or 7.5 mg to 10 mg or 10 mg to 12.5 mg or 12.5 mg to 15 mg or 15 mg to 17.5 mg or 17.5 mg to 20 mg or 20 mg to 22.5 mg or 22.5 mg to 25 mg or 25 mg to 30 mg or 30 mg to 35 mg or 35 mg to 40 mg or 40 mg to 45 mg or 45 mg to 50 mg or 50 mg to 55 mg or 55 mg to 60 mg or 60 mg to 65 mg or 65 mg to 70 mg or 70 mg to 75 mg or 75 mg to 80 mg or 80 mg to 85 mg or 85 mg to 90 mg or 90 mg to 95 mg or 95 mg to 100 mg.

TABLE 1
Stable oral pharmaceutical composition of formula (Ia)

| | Strength - | Strength - |
|---|---|---|
| | 5 mg/ml | 20 mg/ml |
| Ingredients | mg/5 ml | mg/5 ml |
| Compounds of formula (Ia) | 25.00 | 100.00 |
| Sucrose | 3085.66 | 3010.66 |
| Citric Acid, Anhydrous USP | 10.00 | 10.00 |
| Microcrystalline Cellulose and | 164.85 | 164.85 |
| Carboxymethylcellulose Sodium (Avicel CL 611) | | |
| Xanthan Gum NF (Xantural 75) | 6.49 | 6.49 |
| Cherry Flavour | 8.00 | 8.00 |

A suitable thrombopoietin (TPO) receptor agonist Eltrombopag or its pharmaceutically acceptable salts, Avatrombopag or its pharmaceutically acceptable salts and Lusutrombopag or its pharmaceutically acceptable salts.

In a further embodiment, Eltrombopag is administered orally to a subject in an amount of about 1 mg to about 500 mg. In a further embodiment, Avatrombopag is administered orally to a subject in an amount of about 1 mg to 100 mg. In a further embodiment, Lusutrombopag is administered orally to a subject in an amount of about 1 mg to 100 mg.

In a further embodiment, Eltrombopag is administered orally to a subject in an amount of about 12.5 mg, 25 mg, 50 mg, 75 mg and 100 mg. In a further embodiment, Avatrombopag is administered orally to a subject in an amount of about 20 mg. In a further embodiment, Lusutrombopag is administered orally to a subject in an amount of about 3 mg.

In certain embodiments, compounds of formula (Ia) or its pharmaceutically acceptable salts can be further characterized according to the dose of compound administered to a subject, where subject is animal or human.

In some embodiments, compounds of formula (Ia) or its pharmaceutically acceptable salts and a suitable thrombopoietin (TPO) receptor agonist are administered as one composition. In some embodiments, compounds of formula (Ia) or its pharmaceutically acceptable salts and a suitable thrombopoietin (TPO) receptor agonist are administered separately. In some embodiments, compounds of formula (Ia) or its pharmaceutically acceptable salts and a suitable thrombopoietin (TPO) receptor agonist are administered consecutively. In some embodiments, compounds of formula (Ia) or its pharmaceutically acceptable salts and the a suitable thrombopoietin (TPO) receptor agonist are administered simultaneously.

### Parenteral:

The pharmaceutical composition of combination of compound of formula (Ia) or its pharmaceutically acceptable salts and Romiplostim is in parenteral form.

The pharmaceutical composition of combination of compound of formula (Ia) or its pharmaceutically acceptable salts and Romiplostim can be administered via parenteral rout.

In an embodiment, the parenteral formulation of compound of formula (Ia) or its pharmaceutically acceptable salts can be prepared as method known in art.

The parenteral formulation of compound of formula (Ia) or its pharmaceutically acceptable salts comprising one or more pharmaceutically acceptable excipients for use in treating Aplastic anemia (AA).

The pharmaceutically acceptable excipients are selected at least one from dextrose in water, captisol in water, microemulsion and cosolvent or emulsifying agent with pH adjustment for use in treating Aplastic anemia (AA).

Cosolvent or emulsifying agent is selected from Captex™ 300, Solutol™ H15, Propylene glycol, Phospholipon™ 90G, Solutol™ HS15, dimethylacetamide with ethanol, PEG400 and N-methylpyrrolidone.

In certain embodiments, the compound of formula (Ia) or its pharmaceutically acceptable salts, for parenteral administration to a subject at a dose in the range of 1 mg to 50 mg. In a further embodiments, the compound of formula (Ia) or its pharmaceutically acceptable salts, for parenteral administration to a subject at a dose in the range of 1 mg to 2.5 mg or 2.5 mg to 5 mg or 5 mg to 7.5 mg or 7.5 mg to 10 mg or 10 mg to 12.5 mg or 12.5 mg to 15 mg or 15 mg to 17.5 mg or 17.5 mg to 20 mg or 20 mg to 22.5 mg or 22.5 mg to 25 mg or 27.5 mg to 30 mg or 30 mg to 32.5 mg or 32.5 mg to 35 mg or 35 mg to 37.5 mg or 37.5 mg to 40 mg or 40 mg to 42.5 mg or 42.5 mg to 45 mg or 45 mg to 47.5 mg or 47.5 mg to 50 mg. In certain embodiments, the compound of formula (Ia) or a pharmaceutically acceptable salts for parenteral administration to a subject to provide the compound of formula (Ia) or a pharmaceutically acceptable salts at a dose of 0.5 mg, 1 mg, 1.5 mg, 2 mg, 2.5 mg, 3 mg, 2.5 mg, 4 mg, 4.5 mg, 5 mg, 5.5 mg, 6 mg, 6.5 mg, 7 mg, 7.5 mg, 8 mg, 8.5 mg, 9 mg, 9.5 mg and 10 mg to the subject.

The pharmaceutical composition of a suitable thrombopoietin (TPO) receptor agonist comprising one or more pharmaceutically acceptable excipients for use in treating Aplastic anemia (AA).

The pharmaceutical composition as described above is in parenteral form.

A suitable thrombopoietin (TPO) receptor agonist is Romiplostim or its pharmaceutically acceptable salts.

The pharmaceutically acceptable excipients are selected at least one from a buffer, a bulking agent, a stabilizing agent and a surfactant for the treatment of Aplastic Anemia (AA).

Buffer is selected from glycine, histidine, glutamate, succinate, phosphate, acetate and aspartate.

Bulking agent is selected from mannitol, glycine, sucrose, dextran, polyvinylpyrolidone, carboxymethylcellulose, lactose, sorbitol, trehalose or xylitol.

The stabilizing agent is selected from sucrose, trehalose, mannose, maltose, lactose, glucose, raffinose, cellobiose, gentiobiose, isomaltose, arabinose, glucosamine, fructose, mannitol, sorbitol, glycine, arginine HCL, poly-hydroxy compounds, including polysaccharides such as dextran, starch, hydroxyethyl starch, cyclodextrins, N-methylpyrollidene, cellulose and hyaluronic acid, sodium chloride.

The surfactant is selected from sodium lauryl sulfate, dioctyl sodium sulfosuccinate, dioctyl sodium sulfonate, chenodeoxycholic acid, N-lauroylsarcosine sodium salt, lithium dodecyl sulfate, 1-octanesulfonic acid sodium salt, sodium cholate hydrate, sodium deoxycholate, glycodeoxycholic acid sodium salt, benzalkonium chloride or benzethonium chloride, cetylpyridinium chloride monohydrate, hexadecyltrimethylammonium bromide, CHAPS, CHAPSO, SB3-10, SB3-12, digitonin, Triton X-100, Triton X-114, lauromacrogol 400, polyoxyl 40 stearate, polyoxyethylene hydrogenated castor oil 10, 40, 50 and 60, glycerol monostearate, polysorbate 20, 40, 60, 65 and 80, soy lecithin, DOPC, DMPG, DMPC, and DOPG; sucrose fatty acid ester, methyl cellulose and carboxymethyl cellulose.

In a further embodiment, Romiplostim is administered parenterally to a subject in an amount of about 1 μg to about 500 μg, about 50 μg to about 450 μg, about 100 μg to about 400 μg, about 150 μg to about 350 μg, about 200 μg to about 300 μg.

In a certain embodiment, Romiplostim is administered parenterally to a subject in an amount of about 125 μg, 250 μg and 500 μg.

In certain embodiments, compounds of formula (Ia) or its pharmaceutically acceptable salts can be further characterized according to the dose of compound administered to a subject, where subject is animal or human.

In some embodiments, compounds of formula (Ia) or its pharmaceutically acceptable salts and Romiplostim are administered as one composition. In some embodiments, compounds of formula (Ia) or its pharmaceutically acceptable salts and Romiplostim are administered separately. In some embodiments, compounds of formula (Ia) or its pharmaceutically acceptable salts and Romiplostim are administered consecutively. In some embodiments, compounds of formula (Ia) or its pharmaceutically acceptable salts and Romiplostim are administered simultaneously.

Parenteral formulation can be administered by Subcutaneous, Intramuscular, Intravenous and Intrathecal.

Use of Compound of Formula (Ia) or its Pharmaceutically Acceptable Salts in Combination with a Suitable Thrombopoietin (TPO) Receptor Agonist for Use in Treating Aplastic Anemia (AA)

In an embodiment, present invention provides use of combination of compound of formula (Ia) or its pharmaceutically acceptable salts with a suitable thrombopoietin (TPO) receptor agonist for treating Aplastic anemia (AA).

The pharmaceutical acceptable salts of the compound of formula (Ia) may be selected from a suitable inorganic metal salts or organic amines salts.

The inorganic metal salt may be selected from calcium, sodium, potassium, lithium, barium, strontium, magnesium, cesium, copper, cobalt, iron, manganese, lead, aluminum, cadmium, silver, zinc, ammonium and the like.

Organic amines salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, alanine, lysine, arginine, histidine, threonine, proline, glutamine, glycine, 2-thiopheneethanamine, (2S)-3,3-dimethyl-2-butanamine, cyclopentanamine, cycloheptanamine, meglumine, benethamine, dibenzylamine, diphenylamine, α-naphthylamine, 0-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N—N-diethylethanolamine, deanol, hydrabamine, betaine, adamantanamine, L-adamantanmethylamine, tritylamine, glucamine, N-methyl pyrrolidine, urea, procaine, metformin, hexane-1-6-diamine, 2-(2-aminoethoxy)ethanamine, N-methylmorpholine, and N-ethylmorpholine.

A suitable thrombopoietin (TPO) receptor agonist is Romiplostim or its pharmaceutically acceptable salts, Eltrombopag or its pharmaceutically acceptable salts, Avatrombopag or its pharmaceutically acceptable salts and Lusutrombopag or its pharmaceutically acceptable salts. Combination as described above can be administered as oral, rectal, topical, nasal, pulmonary, ocular, intestinal, or parenteral.

In certain embodiments, the compound of formula (Ia) or its pharmaceutically acceptable salts, for oral administration to a subject at a dose in the range of 1 mg to 500 mg or in the range of 50 mg to 450 mg or in the range of 100 mg to 400 mg or in the range of 150 mg to 350 mg or in the range of 200 mg to 300 mg or in the range of 1 mg to 50 mg or in the range of 1 mg to 25 mg to the subject. In certain embodiments, the compound of formula (Ia) or a pharmaceutically acceptable salts for oral administration to a subject to provide the compound of formula (Ta) or a pharmaceutically acceptable salts at a dose of 1 mg to 150 mg. In further embodiments, the compound of formula (Ia) or its pharmaceutically acceptable salts, for oral administration to a subject at a dose in the range of 1 mg to 2.5 mg or 2.5 mg to 5 mg or 5 mg to 7.5 mg or 7.5 mg to 10 mg or 10 mg to 12.5 mg or 12.5 mg to 15 mg or 15 mg to 17.5 mg or 17.5 mg to 20 mg or 20 mg to 22.5 mg or 22.5 mg to 25 mg or 25 mg to 30 mg or 30 mg to 35 mg or 35 mg to 40 mg or 40 mg to 45 mg or 45 mg to 50 mg or 50 mg to 55 mg or 55 mg to 60 mg or 60 mg to 65 mg or 65 mg to 70 mg or 70 mg to 75 mg or 75 mg to 80 mg or 80 mg to 85 mg or 85 mg to 90 mg or 90 mg to 95 mg or 95 mg to 100 mg.

In a further embodiment, Eltrombopag is administered orally to a subject in an amount of about 1 mg to about 500 mg. In a further embodiment, Avatrombopag is administered orally to a subject in an amount of about 1 mg to 100 mg. In a further embodiment, Lusutrombopag is administered orally to a subject in an amount of about 1 mg to 100 mg.

In a further embodiment, Eltrombopag is administered orally to a subject in an amount of about 12.5 mg, 25 mg, 50 mg, 75 mg and 100 mg. In a further embodiment, Avatrombopag is administered orally to a subject in an amount of about 20 mg. In a further embodiment, Lusutrombopag is administered orally to a subject in an amount of about 3 mg.

In certain embodiments, the compound of formula (Ia) or its pharmaceutically acceptable salts, for parenteral administration to a subject at a dose in the range of 1 mg to 50 mg. In a further embodiments, the compound of formula (Ia) or its pharmaceutically acceptable salts, for parenteral administration to a subject at a dose in the range of 1 mg to 2.5 mg or 2.5 mg to 5 mg or 5 mg to 7.5 mg or 7.5 mg to 10 mg or 10 mg to 12.5 mg or 12.5 mg to 15 mg or 15 mg to 17.5 mg or 17.5 mg to 20 mg or 20 mg to 22.5 mg or 22.5 mg to 25 mg or 27.5 mg to 30 mg or 30 mg to 32.5 mg or 32.5 mg to 35 mg or 35 mg to 37.5 mg or 37.5 mg to 40 mg or 40 mg to 42.5 mg or 42.5 mg to 45 mg or 45 mg to 47.5 mg or 47.5 mg to 50 mg. In certain embodiments, the compound of formula (Ia) or a pharmaceutically acceptable salts for parenteral administration to a subject to provide the compound of formula (Ia) or a pharmaceutically acceptable salts at a dose of 0.5 mg, 1 mg, 1.5 mg, 2 mg, 2.5 mg, 3 mg, 2.5 mg, 4 mg, 4.5 mg, 5 mg, 5.5 mg, 6 mg, 6.5 mg, 7 mg, 7.5 mg, 8 mg, 8.5 mg, 9 mg, 9.5 mg and 10 mg to the subject.

In a further embodiment, Romiplostim is administered parenterally to a subject in an amount of about 1 μg to about 500 μg, about 50 μg to about 450 μg, about 100 μg to about 400 μg, about 150 μg to about 350 μg, about 200 μg to about 300 μg.

In a certain embodiment, Romiplostim is administered parenterally to a subject in an amount of about 125 μg, 250 μg and 500 μg. In a further embodiment.

In certain embodiments, combination of compounds of formula (Ia) or its pharmaceutically acceptable salts and a suitable thrombopoietin (TPO) receptor agonist can be further characterized according to the dose of compound administered to a subject, where subject is animal or human.

In some embodiments, compounds of formula (Ia) or its pharmaceutically acceptable salts and the a suitable thrombopoietin (TPO) receptor agonist are administered as one composition. In some embodiments, compounds of formula (Ia) or its pharmaceutically acceptable salts and a suitable thrombopoietin (TPO) receptor agonist are administered separately. In some embodiments, compounds of formula (Ia) or its pharmaceutically acceptable salts and a suitable thrombopoietin (TPO) receptor agonist are administered consecutively. In some embodiments, compounds of formula (Ia) or its pharmaceutically acceptable salts and the a suitable thrombopoietin (TPO) receptor agonist are administered simultaneously.

### Method of Treating Aplastic Anemia (AA) by Compound of Formula (Ia)

In an embodiment the present invention provides a method of treating Aplastic anemia (AA) in a subject, comprising administering an effective amount of compound of formula (Ia) or its pharmaceutically acceptable salts.

The pharmaceutical acceptable salts of the compound of formula (Ia) may be selected from a suitable inorganic metal salts or organic amines salts.

The inorganic metal salt may be selected from calcium, sodium, potassium, lithium, barium, strontium, magnesium, cesium, copper, cobalt, iron, manganese, lead, aluminum, cadmium, silver, zinc, ammonium and the like.

Organic amines salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, alanine, lysine, arginine, histidine, threonine, proline, glutamine, glycine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N—N-diethylethanolamine, deanol, hydrabamine, betaine, adamantanamine, L-adamantanmethylamine, tritylamine, glucamine, N-methyl pyrrolidine, urea, procaine, metformin, hexane-1-6-diamine, 2-(2-aminoethoxy)ethanamine, N-methylmorpholine, and N-ethylmorpholine.

In certain embodiments, the compound of formula (Ia) or its pharmaceutically acceptable salts, for administration to a subject at a dose in the range of 1 mg to 500 mg or in the range of 50 mg to 450 mg or in the range of 100 mg to 400 mg or in the range of 150 mg to 350 mg or in the range of 200 mg to 300 mg or in the range of 1 mg to 50 mg or in the range of 1 mg to 25 mg to the subject. In certain embodiments, the compound of formula (Ia) or a pharmaceutically acceptable salts for administration to a subject to provide the compound of formula (Ta) or a pharmaceutically acceptable salts at a dose of 1 mg to 150 mg. In further embodiments, the compound of formula (Ia) or its pharmaceutically acceptable salts, for administration to a subject at a dose in the range of 1 mg to 2.5 mg or 2.5 mg to 5 mg or 5 mg to 7.5 mg or 7.5 mg to 10 mg or 10 mg to 12.5 mg or 12.5 mg to 15 mg or 15 mg to 17.5 mg or 17.5 mg to 20 mg or 20 mg to 22.5 mg or 22.5 mg to 25 mg or 25 mg to 30 mg or 30 mg to 35 mg or 35 mg to 40 mg or 40 mg to 45 mg or 45 mg to 50 mg or 50 mg to 55 mg or 55 mg to 60 mg or 60 mg to 65 mg or 65 mg to 70 mg or 70 mg to 75 mg or 75 mg to 80 mg or 80 mg to 85 mg or 85 mg to 90 mg or 90 mg to 95 mg or 95 mg to 100 mg.

In certain embodiments, compounds of formula (Ia) or its pharmaceutically acceptable salts can be further characterized according to the dose of compound administered to a subject, where subject is animal or human.

The composition of formula (Ia) or its pharmaceutically acceptable salts can be prepared as anywhere disclosed in the specification.

Combination of Compound of Formula (Ia) or its Pharmaceutically Acceptable Salts and a Suitable Thrombopoietin (TPO) Receptor Agonist Optionally with Cyclosporine

A suitable thrombopoietin (TPO) receptor agonist is Eltrombopag or its pharmaceutically acceptable salts, Romiplostim or its pharmaceutically acceptable salts, Avatrombopag or its pharmaceutically acceptable salts and Lusutrombopag or its pharmaceutically acceptable salts.

The pharmaceutical acceptable salts of the compound of formula (Ia) may be selected from a suitable inorganic metal salt or organic amines salts.

The inorganic metal salt may be selected from calcium, sodium, potassium, lithium, barium, strontium, magnesium, cesium, copper, cobalt, iron, manganese, lead, aluminum, cadmium, silver, zinc, ammonium and the like.

Organic amines salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, alanine, lysine, arginine, histidine, threonine, proline, glutamine, glycine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N—N-diethylethanolamine, deanol, hydrabamine, betaine, adamantanamine, L-adamantanmethylamine, tritylamine, glucamine, N-methyl pyrrolidine, urea, procaine, metformin, hexane-1-6-diamine, 2-(2-aminoethoxy)ethanamine, N-methylmorpholine, and N-ethylmorpholine.

In an embodiment, present invention provides a pharmaceutical composition comprising compound of formula (Ia) or its pharmaceutically acceptable salts and a suitable thrombopoietin (TPO) receptor agonist optionally with cyclosporine.

The pharmaceutical composition, dosage form and rout of administration as described above or anywhere in the specification.

The compound of formula (Ia) may be prepared by any of the methods known in the art including those processes disclosed in the prior art such as those mentioned elsewhere in the specification. Romiplostim may be prepared by any of the methods known in the art including those processes disclosed in the prior art such as those mentioned elsewhere in the specification.

### EXAMPLES

The effect of combination of compounds of formula (Ia) and a suitable thrombopoietin (TPO) receptor agonist in animal models for Aplastic Anemia (AA) is shown herein below.

### Animal Models of Aplastic Anemia (AA)

### Example 1: Effect of Formula (Ia), Romiplostim and its Combination on Busulphan-Induced Aplastic Anemia in Balb/c Mice

Aplastic anemia (AA) was induced in BALB/c mice by administration of busulphan (BU). BU was dissolved in acetone at concentrations of 5-10 mg/mL. Immediately before administration, deionized water was added to the BU-acetone solution and the solution was given by intraperitoneal (ip) injection at dose of 9 mg/kg. Control mice were given vehicle at the same dose volume.⁹ Compound of formula (Ia) (15 mg/kg, PO, daily) and Romiplostim (50 μg/kg, SC, once a week) combination were administered.

Mice were be dosed with vehicle or BU on 10 occasions over a period of 21 days (days 1, 3, 6, 8, 10, 13, 15, 17, 19 and 21). Between 1 to 127 days after the final BU dose, mice from each group were autopsied for blood.⁹

The changes in the white blood cell (WBC) and platelet count were measured after 44 days of treatment. Data are mean±SEM (n=6 for each group).

TABLE 2
The effect of formula (Ia) alone, Romiplostim alone and its combination on white blood
cell (WBC) and platelets in busulphan-induced aplastic anemia in Balb/c mice.

| | | | | | | % Change | | | | | | % Change | | |
|---|---|---|---|---|---|---|---|---|---|---|---|---|---|---|
| | | | WBC | | | against vehicle | | | Platelets | | | against vehicle | | |
| Treatment | Dose | | (10\hat{ }3/uL) | | | control | | | (10\hat{ }3/uL) | | | control | | |
| Normal control | NA | | 5.5 | ± | 0.7 | 134.7 | ± | 29.1 | 1261.7 | ± | 100.3 | 143.6 | ± | 19.4 |
| Vehicle control | 10 | ml/kg | 2.4 | ± | 0.3 | 0.0 | ± | 13.4 | 517.8 | ± | 70.5 | 0.0 | ± | 13.6 |
| Formula (Ia) | (15 | mg/kg) | 2.6 | ± | 0.2 | 10.9 | ± | 10.4 | 676.2 | ± | 80.1 | 30.6 | ± | 15.5 |
| Romiplostim | (50 | μg/kg) | 4.2 | ± | 0.5 | 79.3 | ± | 23.2 | 1257.0 | ± | 57.0 | 142.7 | ± | 11.0 |

| | | | | | | | | | | | | | |
|---|---|---|---|---|---|---|---|---|---|---|---|---|---|
| Formula (Ia) + | (15 mg/kg) + | 5.0 | ± | 0.4 | 113.4 | ± | 16.0 | 1330.0 | ± | 89.7 | 156.8 | ± | 17.3 |
| Romiplostim | (50 μg/kg) | | | | | | | | | | | | |

#P < 0.05 against normal control and *P < 0.05 against vehicle control was considered significant.
Conclusion: Combination of Formula (Ia) and Romiplostim significantly increased two lineages of hematopoiesis in busulphan- induced aplastic anemia in Balb/c mice.

### Example 2: Effect of Formula (Ia) and its Combination with Romiplostim on Cyclophosphamide-Induced Aplastic Anemia in C57 Mice

Aplastic anemia (AA) was induced in C57 mice by administration of cyclophosphamide. Cyclophosphamide was dissolved in deionized water at concentrations of 20 mg/mL. Immediately before administration, deionized water was added to the stock solution and the solution was given by intraperitoneal (ip) injection at dose of 100 mg/kg. Mice were dosed with vehicle or cyclophosphamide for 4 days. Control mice were given vehicle at the same dose volume.¹⁰ Compound of formula (Ia) (15 mg/kg, PO, daily) and Romiplostim (30 μg/kg, SC, once a week) combination were administered.

Blood was collected from each group after 11 days of treatment. The changes in the white blood cell (WBC) and platelet count were measured. Data are mean±SEM (n=6 for each group).

TABLE 3
The effect of formula (Ia) and its combination with Romiplostim on white blood cell
(WBC) and platelets in cyclophosphamide-induced aplastic anemia in C57 mice.

| | | | | | | % Change | | | | | | % Change | | |
|---|---|---|---|---|---|---|---|---|---|---|---|---|---|---|
| | | | WBC | | | against vehicle | | | Platelets | | | against vehicle | | |
| Treatment | Dose | | (10\hat{ }3/uL) | | | control | | | (10\hat{ }3/uL) | | | control | | |
| Normal control | NA | | 9.2 | ± | 0.6 | 44.7 | ± | 9.3 | 1143.2 | ± | 28.8 | 103.5 | ± | 5.1 |
| Vehicle control | (10 | ml/kg) | 6.4 | ± | 0.5 | 0.0 | ± | 8.1 | 561.8 | ± | 83.2 | 0.0 | ± | 14.8 |
| Formula (Ia) | (15 | mg/kg) | 7.0 | ± | 1.0 | 10.1 | ± | 15.6 | 742.3 | ± | 31.0 | 32.1 | ± | 5.5 |

| | | | | | | | | | | | | | |
|---|---|---|---|---|---|---|---|---|---|---|---|---|---|
| Formula (Ia) + | (15 mg/kg) + | 8.7 | ± | 0.5 | 37.2 | ± | 8.4 | 1334.2 | ± | 113.1 | 137.5 | ± | 20.1 |
| Romiplostim | (30 μg/kg) | | | | | | | | | | | | |

#P < 0.05 against normal control and *P < 0.05 against vehicle control was considered significant.
Conclusion: Combination of Formula (Ia) and Romiplostim significantly increased two lineages hematopoiesis in cyclophosphamide- induced aplastic anemia in C57 mice.

### REFERENCES

1. Dezern A E, Brodsky R A. Clinical management of aplastic anemia. Expert Rev Hematol. 2011 April; 4(2):221-30. doi: 10.1586/ehm.11.11. PMID: 21495931; PMCID: PMC3138728.
2. Young N S. Aplastic Anemia. N Engl J Med. 2018 Oct. 25; 379(17):1643-1656. doi: 10.1056/NEJMra1413485. PMID: 30354958; PMCID: PMC6467577.
3. Peslak S A, Olson T, Babushok D V. Diagnosis and Treatment of Aplastic Anemia. Curr Treat Options Oncol. 2017 Nov. 16; 18(12):70. doi: 10.1007/s11864-017-0511-z. PMID: 29143887; PMCID: PMC5804354.
4. Olcay L, Yetgin S, Aoshima H. Disorders mimicking myelodysplastic syndrome and difficulties in its diagnosis. IntechOpen; 2016 Sep. 7.
5. Desmond R, Townsley D M, Dunbar C, Young N S. Eltrombopag in aplastic anemia. Semin Hematol. 2015 January; 52(1):31-7. doi: 10.1053/j.seminhematol.2014.10.002. Epub 2014 Oct. 31. PMID: 25578417; PMCID: PMC4293077.
6. Lum S H, Grainger J D. Eltrombopag for the treatment of aplastic anemia: current perspectives. Drug Des Devel Ther. 2016 Sep. 13; 10:2833-2843. doi: 10.2147/DDDT.S95715. PMID: 27695288; PMCID: PMC5028092.
7. Al-Samkari H, Kuter D J. Optimal use of thrombopoietin receptor agonists in immune thrombocytopenia. Ther Adv Hematol. 2019 Apr. 11; 10:2040620719841735. doi: 10.1177/2040620719841735. PMID: 31007886; PMCID: PMC6460888.
8. Joharapurkar A A, Patel V J, Kshirsagar S G, Patel M S, Savsani H H, Kajavadara C, Valani D, Jain M R. Prolyl hydroxylase inhibitor desidustat improves anemia in erythropoietin hyporesponsive state. Curr Res Pharmacol Drug Discov. 2022 Apr. 30; 3:100102. doi: 10.1016/j.crphar.2022.100102. PMID: 35570856; PMCID: PMC9096675.
9. Turton J A, Sones W R, Andrews C M, Pilling A M, Williams T C, Molyneux G, Rizzo S, Gordon-Smith E C, Gibson F M. Further development of a model of chronic bone marrow aplasia in the busulphan-treated mouse. International journal of experimental pathology. 2006 February; 87(1):49-63.
10. Lu W, Jia D, An S, Mu M, Qiao X, Liu Y, Li X, Wang D. Calf Spleen Extractive Injection protects mice against cyclophosphamide-induced hematopoietic injury through G-CSF-mediated JAK2/STAT3 signaling. Sci Rep. 2017 Aug. 21; 7(1):8402. doi: 10.1038/s41598-017-08970-3. PMID: 28827748; PMCID: PMC5566473.

1-3. (canceled)

4. A combination of compound of formula (Ia) or its pharmaceutically acceptable salts with a suitable thrombopoietin (TPO) receptor agonist, wherein formula (Ia) is represented by:

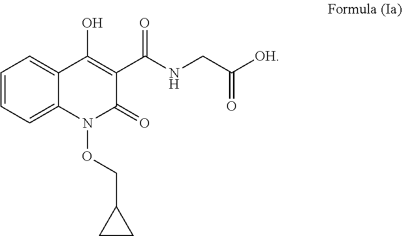 [SMILES: O=C(O)CNC(=O)c1c(O)c2ccccc2n(OCC2CC2)c1=O]

5. A combination as claimed in claim 4, wherein the pharmaceutical acceptable salts of the compound of formula (Ia) is selected from a suitable inorganic metal salts or organic amines salts; wherein the inorganic metal salt is selected from calcium, sodium, potassium, lithium, barium, strontium, magnesium, cesium, copper, cobalt, iron, manganese, lead, aluminum, cadmium, silver, zinc, ammonium; the organic amine salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, alanine, lysine, arginine, histidine, threonine, proline, glutamine, glycine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N—N-diethylethanolamine, deanol, hydrabamine, betaine, adamantanamine, L-adamantanmethylamine, tritylamine, glucamine, N-methyl pyrrolidine, urea, procaine, metformin, hexane-1-6-diamine, 2-(2-aminoethoxy)ethanamine, N-methylmorpholine, and N-ethylmorpholine.

6. A combination as claimed in claim 4, wherein a suitable thrombopoietin (TPO) receptor agonist is selected from Eltrombopag or its pharmaceutically acceptable salts, Romiplostim or its pharmaceutically acceptable salts, Avatrombopag or its pharmaceutically acceptable salts and Lusutrombopag or its pharmaceutically acceptable salts.

7. A combination as claimed in claim 4 can be administered orally or parenterally.

8. A combination as claimed in claim 4, wherein the compound of formula (Ia) or its pharmaceutically acceptable salts is administered orally to a subject in amount of 1 mg to 500 mg or 50 mg to 450 mg or 100 mg to 400 mg or 150 mg to 350 mg or 200 mg to 300 mg or 1 mg to 50 mg or 1 mg to 25 mg to the subject, preferably wherein the compound of formula (Ia) or its pharmaceutically acceptable salts is administered orally to a subject at a dose of 1 mg to 2.5 mg or 2.5 mg to 5 mg or 5 mg to 7.5 mg or 7.5 mg to 10 mg or 10 mg to 12.5 mg or 12.5 mg to 15 mg or 15 mg to 17.5 mg or 17.5 mg to 20 mg or 20 mg to 22.5 mg or 22.5 mg to 25 mg or 25 mg to 30 mg or 30 mg to 35 mg or 35 mg to 40 mg or 40 mg to 45 mg or 45 mg to 50 mg or 50 mg to 55 mg or 55 mg to 60 mg or 60 mg to 65 mg or 65 mg to 70 mg or 70 mg to 75 mg or 75 mg to 80 mg or 80 mg to 85 mg or 85 mg to 90 mg or 90 mg to 95 mg or 95 mg to 100 mg.

9. (canceled)

10. A combination as claimed in claim 4, wherein Eltrombopag is administered orally to a subject in an amount of 1 mg to 500 mg, Avatrombopag is administered orally to a subject in an amount of 1 mg to 100 mg and Lusutrombopag is administered orally to a subject in an amount of 1 mg to 100 mg, preferably wherein Eltrombopag is administered orally to a subject in an amount of 12.5 mg, 25 mg, 50 mg, 75 mg and 100 mg, Avatrombopag is administered orally to a subject in an amount of 20 mg and Lusutrombopag is administered orally to a subject in an amount of 3 mg.

11. (canceled)

12. A combination as claimed in claim 4, wherein the compound of formula (Ia) or its pharmaceutically acceptable salts is administered parenterally to a subject in amount of 1 mg to 2.5 mg or 2.5 mg to 5 mg or 5 mg to 7.5 mg or 7.5 mg to 10 mg or 10 mg to 12.5 mg or 12.5 mg to 15 mg or 15 mg to 17.5 mg or 17.5 mg to 20 mg or 20 mg to 22.5 mg or 22.5 mg to 25 mg or 27.5 mg to 30 mg or 30 mg to 32.5 mg or 32.5 mg to 35 mg or 35 mg to 37.5 mg or 37.5 mg to 40 mg or 40 mg to 42.5 mg or 42.5 mg to 45 mg or 45 mg to 47.5 mg or 47.5 mg to 50 mg, preferably wherein the compound of formula (Ia) or its pharmaceutically acceptable salts is administered parenterally to a subject in amount of 0.5 mg, 1 mg, 1.5 mg, 2 mg, 2.5 mg, 3 mg, 2.5 mg, 4 mg, 4.5 mg, 5 mg, 5.5 mg, 6 mg, 6.5 mg, 7 mg, 7.5 mg, 8 mg, 8.5 mg, 9 mg, 9.5 mg and 10 mg.

13. (canceled)

14. A combination as claimed in claim 4, wherein thrombopoietin (TPO) receptor agonist which is Romiplostim, administered parenterally to a subject in an amount of 1 μg to 500 μg, 50 μg to 450 μg, 100 μg to 400 μg, 150 μg to 350 μg, 200 μg to 300 μg, preferably wherein thrombopoietin (TPO) receptor agonist which is Romiplostim, administered parenterally to a subject in an amount of 125 μg, 250 μg and 500 μg.

15. (canceled)

16. A combination as claimed in claim 4, wherein combination of the compound of formula (Ia) or its pharmaceutically acceptable salts and a suitable thrombopoietin (TPO) receptor agonist is administered to a subject, where subject is animal or human.

17. A combination as claimed in claim 4, wherein the compound of formula (Ia) or its pharmaceutically acceptable salts and a suitable thrombopoietin (TPO) receptor agonist is administered as one composition, separately, consecutively or simultaneously.

18. A method of treating Aplastic anemia (AA) in a subject, comprising administering a combination of compound of formula (Ia) or its pharmaceutically acceptable salts with a suitable thrombopoietin (TPO) receptor agonist, wherein formula (Ia) is

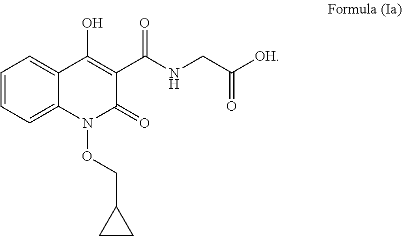 [SMILES: O=C(O)CNC(=O)c1c(O)c2ccccc2n(OCC2CC2)c1=O]

19. A method of treating Aplastic anemia (AA) in a subject as claimed in claim 18, wherein the pharmaceutical acceptable salts of the compound of formula (Ta) is selected from suitable inorganic metal salts or organic amines salts; wherein the inorganic metal salt is selected from calcium, sodium, potassium, lithium, barium, strontium, magnesium, cesium, copper, cobalt, iron, manganese, lead, aluminum, cadmium, silver, zinc, ammonium; the organic amine salt is selected from methylamine, dimethylamine, ethylamine, diethyl amine, n-propyl amine, isopropyl amine, diisopropyl amine, N-methyl isopropyl amine, n-butyl amine, t-butyl amine, 2-butamine, 1,2-ethane diamine, N-methylglucamine, N,N,N-trimethyl ethanolamine hydroxide (choline), tromethamine, cyclohexylamine, N-methyl cyclohexylamine, guanidine, N-(4-aminobutyl) guanidine, dicyclohexylamine, benzene-methanamine, ethanolamine, diethanolamine, tris-(hydroxymethyl)methylamine, hydroxylamine, methanaminium, benzylamine, N-methylbenzylamine, N-ethyl benzylamine, 4-methoxybenzylamine, pyrrolidine, piperidine, piperazine, morpholine, 2-aminopyrimidine, alanine, lysine, arginine, histidine, threonine, proline, glutamine, glycine, 2-thiopheneethanamine, (2S)-3,3-dimethyl-2-butanamine, cyclopentanamine, cycloheptanamine, meglumine, benethamine, dibenzylamine, diphenylamine, α-naphthylamine, O-phenylenediamine, 1,3-Diaminopropane, (S)-α-naphthylethylamine, (S)-3-methoxyphenylethylamine, (S)-4-methoxyphenylethylamine, (S)-4-chlorophenylethylamine, (S)-4-methylphenylethylamine, cinchonine, cinchonidine, (−)-quinine, triethanolamine, imidazole, ethylenediamine, epolamine, morpholine 4-(2-hydroxyethyl), N—N-diethylethanolamine, deanol, hydrabamine, betaine, adamantanamine, L-adamantanmethylamine, tritylamine, glucamine, N-methyl pyrrolidine, urea, procaine, metformin, hexane-1-6-diamine, 2-(2-aminoethoxy)ethanamine, N-methylmorpholine, and N-ethylmorpholine.

20. A method of treating Aplastic anemia (AA) in a subject as claimed in claim 18, wherein a suitable thrombopoietin (TPO) receptor agonist is selected from Romiplostim or its pharmaceutically acceptable salts, Eltrombopag or its pharmaceutically acceptable salts, Avatrombopag or its pharmaceutically acceptable salts and Lusutrombopag or its pharmaceutically acceptable salts.

21. A method of treating Aplastic anemia (AA) in a subject as claimed in claim 11, wherein combination can be administered orally or parenterally.

22. A method of treating Aplastic anemia (AA) in a subject as claimed in claim 18, wherein the compound of formula (Ia) or its pharmaceutically acceptable salts is administered orally to a subject in amount of 1 mg to 500 mg, 50 mg to 450 mg, 100 mg to 400 mg, 150 mg to 350 mg, 200 mg to 300 mg, 1 mg to 50 mg, 1 mg to 25 mg to the subject, preferably wherein the compound of formula (Ia) or its pharmaceutically acceptable salts is administered orally to a subject at a dose of 1 mg to 2.5 mg or 2.5 mg to 5 mg or 5 mg to 7.5 mg or 7.5 mg to 10 mg or 10 mg to 12.5 mg or 12.5 mg to 15 mg or 15 mg to 17.5 mg or 17.5 mg to 20 mg or 20 mg to 22.5 mg or 22.5 mg to 25 mg or 25 mg to 30 mg or 30 mg to 35 mg or 35 mg to 40 mg or 40 mg to 45 mg or 45 mg to 50 mg or 50 mg to 55 mg or 55 mg to 60 mg or 60 mg to 65 mg or 65 mg to 70 mg or 70 mg to 75 mg or 75 mg to 80 mg or 80 mg to 85 mg or 85 mg to 90 mg or 90 mg to 95 mg or 95 mg to 100 mg.

23. (canceled)

24. A method of treating Aplastic anemia (AA) in a subject as claimed in claim 18, wherein Eltrombopag is administered orally to a subject in an amount of 1 mg to 500 mg, Avatrombopag is administered orally to a subject in an amount of 1 mg to 100 mg and Lusutrombopag is administered orally to a subject in an amount of 1 mg to 100 mg, preferably wherein Eltrombopag is administered orally to a subject in an amount of 12.5 mg, 25 mg, 50 mg, 75 mg and 100 mg, Avatrombopag is administered orally to a subject in an amount of 20 mg and Lusutrombopag is administered orally to a subject in an amount of 3 mg.

25. (canceled)

26. A method of treating Aplastic anemia (AA) in a subject as claimed in claim 18, wherein the compound of formula (Ia) or its pharmaceutically acceptable salts is administered parenterally to a subject in amount of 1 mg to 2.5 mg or 2.5 mg to 5 mg or 5 mg to 7.5 mg or 7.5 mg to 10 mg or 10 mg to 12.5 mg or 12.5 mg to 15 mg or 15 mg to 17.5 mg or 17.5 mg to 20 mg or 20 mg to 22.5 mg or 22.5 mg to 25 mg or 27.5 mg to 30 mg or 30 mg to 32.5 mg or 32.5 mg to 35 mg or 35 mg to 37.5 mg or 37.5 mg to 40 mg or 40 mg to 42.5 mg or 42.5 mg to 45 mg or 45 mg to 47.5 mg or 47.5 mg to 50 mg, preferably wherein the compound of formula (Ia) or its pharmaceutically acceptable salts is administered parenterally to a subject in amount of 0.5 mg, 1 mg, 1.5 mg, 2 mg, 2.5 mg, 3 mg, 2.5 mg, 4 mg, 4.5 mg, 5 mg, 5.5 mg, 6 mg, 6.5 mg, 7 mg, 7.5 mg, 8 mg, 8.5 mg, 9 mg, 9.5 mg and 10 mg.

27. (canceled)

28. A method of treating Aplastic anemia (AA) in a subject as claimed in claim 18, wherein thrombopoietin (TPO) receptor agonist which is Romiplostim, administered parenterally to a subject in an amount of 1 μg to 500 μg, 50 μg to 450 μg, 100 μg to 400 μg, 150 μg to 350 μg, 200 μg to 300 μg, preferably wherein thrombopoietin (TPO) receptor agonist which is Romiplostim, administered parenterally to a subject in an amount of 125 μg, 250 μg and 500 g.

29. (canceled)

30. A method of treating Aplastic anemia (AA) in a subject as claimed in claim 18, wherein combination of the compound of formula (Ia) or its pharmaceutically acceptable salts and a suitable thrombopoietin (TPO) receptor agonist is administered to a subject, where subject is animal or human.

31. A method of treating Aplastic anemia (AA) in a subject as claimed in claim 18, wherein the compound of formula (Ia) or its pharmaceutically acceptable salts and a suitable thrombopoietin (TPO) receptor agonist is administered as one composition, separately, consecutively or simultaneously.

32-70. (canceled)
